(19) United States
(12) Patent Application Publication
(10) Pub. No.: US 2018/0103881 A1
(43) Pub. Date: Apr. 19, 2018

(54) ELECTRONIC APPARATUS AND METHOD FOR PROVIDING BLOOD SUGAR CARE SERVICE

(71) Applicant: Samsung Electronics Co., Ltd., Suwon-si (KR)

(72) Inventors: Hyoung-seon CHOI, Seoul (KR); Seo-young YOON, Suwon-si (KR); Chul-ho CHO, Yongin-si (KR); Seong-je CHO, Suwon-si (KR)

(21) Appl. No.: 15/617,436

(22) Filed: Jun. 8, 2017

(30) Foreign Application Priority Data
Oct. 19, 2016 (KR) 10-2016-0136002

(51) Int. Cl. A61B 5/145; A61B 5/00; A61B 5/11

(52) U.S. Cl. CPC A61B 5/14532; A61B 5/7275; A61B 5/1118; A61B 5/7445; A61B 5/0004; A61B 5/7405; A61B 5/748; A61B 5/4866

(57) ABSTRACT

An apparatus and a method for providing blood sugar care service are provided. The method includes obtaining blood sugar information of a user, when the blood sugar information is obtained for a predetermined time unit, generating a blood sugar profile based on the blood sugar information obtained during the predetermined time unit, comparing the generated blood sugar profile and a previously generated blood sugar profile, setting a blood sugar profile proximate to a normal blood sugar range as a target profile, and providing a state of blood sugar of the user based on the set target profile, thereby providing a customized blood sugar care state suitable to each user in consideration of the state of blood sugar of individuals.

### CROSS-REFERENCE TO RELATED APPLICATION(S)

This application claims the benefit under 35 U.S.C. § 119(a) of a Korean patent application filed on Oct. 19, 2016 in the Korean Intellectual Property Office and assigned Serial No. 10-2016-0136002, the entire disclosure of which is hereby incorporated by reference.

### TECHNICAL FIELD

The present disclosure relates to an electronic apparatus and method for providing blood sugar care service. More particularly, the present disclosure relates to an electronic apparatus and method for providing blood sugar care service based on a user's measured blood sugar information.

### BACKGROUND

Blood sugar care services of the related art provide states of blood sugar care based on uniform normal blood sugar levels to not only normal people but also to people having high or low blood sugar levels.

When such blood sugar care services are provided, people with normal blood sugar levels may also be measured to have high blood sugar levels when their blood sugar levels are measured right after eating food, providing a blood sugar care state where high blood sugar level risks may occur.

Not only that, blood sugar care services of the related art are provided based on uniform normal blood sugar levels, and thus people having high blood sugar levels or low blood sugar levels, for whom it is difficult to approach normal blood sugar levels, will continue to receive results showing that they continue to have high blood sugar levels or low blood sugar levels and thus care is required.

When these people keep receiving such results, there is a problem that people having high blood sugar levels or low blood sugar levels, for whom it is difficult to approach normal blood sugar levels, might lose their will to take care of their blood sugar levels.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide a customized blood sugar care state that is suitable to each user by taking into account the blood sugar state of individuals.

In accordance with an aspect of the present disclosure, a method for providing blood sugar care service in an electronic apparatus is provided. The method includes obtaining blood sugar information of a user, when the blood sugar information is obtained for a predetermined time unit, generating a blood sugar profile based on the blood sugar information obtained during the predetermined time unit, comparing the generated blood sugar profile and a previously generated blood sugar profile, setting a blood sugar profile proximate to a normal blood sugar range as a target profile, and providing a state of blood sugar of the user based on the set target profile.

Further, the setting the target profile may include computing a blood sugar care evaluation value corresponding to each of the generated blood sugar profile and the previously generated blood sugar profile, according to a predetermined condition, and determining the blood sugar profile having a higher value, of the computed blood sugar care evaluation values, as the blood sugar profile proximate to the predetermined normal blood sugar range, and setting the determined blood sugar profile as the target profile.

Further, the computing may compute the blood sugar care evaluation value of the blood sugar profile based on at least one of an extent to which the blood sugar profile is included in the normal blood sugar range, a difference between a maximum blood sugar value and a minimum blood sugar value based on a plurality of blood sugar information included in the blood sugar profile, or a period of time the blood sugar information is maintained outside the normal blood sugar range based on the plurality of blood sugar information included in the blood sugar profile.

Further, when the blood sugar information is obtained after the target profile is set, the providing may compare the blood sugar information at the time point the blood sugar information was received, of the plurality of blood sugar information constituting the target profile, and the received blood sugar information, and provide a current blood sugar state of the user.

Further, when the blood sugar profile is generated based on the obtained blood sugar information after the target profile is set, the providing may analyze the generated blood sugar profile based on at least one of the target profile and the normal blood sugar range and provide a state of blood sugar care of the user by period.

Further, the providing may provide the state of blood sugar care of the user by period based on an extent where the generated blood sugar profile belongs to the normal blood sugar range, of a plurality of predetermined evaluation grade sections, or the evaluation grade section to which an error extent between the generated blood sugar profile and the target profile belongs.

The method may further comprise generating a blood sugar history profile based on a plurality of blood sugar information included in each of a plurality of blood sugar profiles generated in the predetermined time unit, wherein the providing determines a section that exists outside the normal blood sugar range based on the blood sugar history profile, and predicts a period where the user's blood sugar will be outside the normal blood sugar range based on the determined section, and provides the predicted period.

The method may further include, when activity information including at least one of food information and exercise information of the user is input, storing the activity information and the blood sugar information measured at the time point the activity information was generated, wherein, when the blood sugar information measured of the user is obtained after the target profile is generated, the providing provides the pre-stored activity information based on the blood sugar information of the point where the obtained blood sugar information is located on the target profile.

Further, the method may include transmitting the blood sugar profile to an external server, and, when the external server generated and transmitted ranking information of a blood sugar care group to which the user belongs based on the blood sugar profile, receiving and providing the ranking information.

In accordance with another aspect of the present disclosure, an electronic apparatus is provided. The electronic apparatus includes a blood sugar measurer configured to measure blood sugar of a user, an outputter configured to output a state of blood sugar of the user, and a processor configured, when blood sugar information measured by the blood sugar measurer is obtained during a predetermined time unit, to generate a blood sugar profile based on the blood sugar information obtained during the predetermined time unit, compare the generated blood sugar profile and a previously generated blood sugar profile, set a blood sugar profile proximate to a normal blood sugar range as a target profile, and control the outputter to output a state of blood sugar of the user based on the set target profile.

Further, the processor may compute a blood sugar care evaluation value corresponding to each of the generated blood sugar profile and the previously generated blood sugar profile according to a predetermined condition, determine the blood sugar profile having a higher value, of the computed blood sugar care evaluation values, as the blood sugar profile proximate to the predetermined normal blood sugar range, and set the determined blood sugar profile as the target profile.

Further, the processor may compute the blood sugar care evaluation value of the blood sugar profile based on at least one of an extent to which the blood sugar profile is included in the normal blood sugar range, a difference between a maximum blood sugar value and a minimum blood sugar value based on a plurality of blood sugar information included in the blood sugar profile, or a period of time the blood sugar information is maintained outside the normal blood sugar range based on the plurality of blood sugar information included in the blood sugar profile.

Further, when the blood sugar information is obtained after the target profile is set, the processor may compare the blood sugar information at the time point the blood sugar information was received, of the plurality of blood sugar information constituting the target profile, and the received blood sugar information, and control the outputter to provide a current blood sugar state of the user.

Further, when the blood sugar profile is generated based on the obtained blood sugar information after the target profile is set, the processor may control the outputter to analyze the generated blood sugar profile based on at least one of the target profile and the normal blood sugar range and output a state of blood sugar care of the user by period.

Further, the processor may provide the state of blood sugar of the user by period based on an extent to which the generated blood sugar profile belongs to the normal blood sugar range of a plurality of predetermined evaluation grade sections or the evaluation grade section to which an error extent between the generated blood sugar profile and the target profile belongs.

Further, the processor may generate a blood sugar history profile based on a plurality of blood sugar information included in each of a plurality of blood sugar profiles generated in the predetermined time unit, determine a section that exists outside the normal blood sugar range based on the blood sugar history profile, predict a period where the user's blood sugar will be outside the normal blood sugar range based on the determined section, and control the outputter to output the predicted result information.

The electronic apparatus may include a storage, and, when activity information including at least one of food information and exercise information of the user is input, the processor may control the storage to store the activity information and the blood sugar information measured at a time point the activity information was generated, and, when the blood sugar information measured of the user is obtained after the target profile is generated, control the outputter to output the activity information stored in the storage based on the blood sugar information at a point where the obtained blood sugar information is located on the target profile.

Further, the electronic apparatus may include a communicator configured to transmit the blood sugar profile to an external server, and, when the external server generated and transmitted ranking information of a blood sugar care group to which the user belongs based on the blood sugar profile, the processor controls the outputter to output the received ranking information when the ranking information is received.

In accordance with another aspect of the present disclosure, a computer readable record medium storing a program for executing the following operations in combination with an electronic apparatus is provided. The operations include obtaining blood sugar information of a user, when the blood sugar information is obtained during a predetermined time unit, generating a blood sugar profile based on the blood sugar information obtained during the predetermined time unit, comparing the generated blood sugar profile and a previously generated blood sugar profile, setting a blood sugar profile proximate to a normal blood sugar range as a target profile, and providing a state of blood sugar of the user based on the set target profile.

According to the aforementioned various embodiments of the present disclosure, the electronic apparatus may provide a customized blood sugar care state, that is suitable to a user, by taking into account the blood sugar state of individuals.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1A is a schematic block diagram of an electronic apparatus that measures and cares a user's blood sugar according to an embodiment of the present disclosure;

FIG. 1B is a schematic block diagram of an electronic apparatus that cares a user's blood sugar according to an embodiment of the present disclosure;

FIG. 2 is a block diagram of an electronic apparatus according to an embodiment of the present disclosure;

FIG. 3 is a view for setting a target profile in an electronic apparatus according to an embodiment of the present disclosure;

FIG. 4A is a first view for providing a blood sugar state of a user based on a predetermined target profile in an electronic apparatus according to an embodiment of the present disclosure;

FIG. 4B is a second view for providing a blood sugar state of a user based on a predetermined target profile in an electronic apparatus according to an embodiment of the present disclosure;

FIG. 5 is a third view for providing a blood sugar state of a user based on a predetermined target profile in an electronic apparatus according to an embodiment of the present disclosure;

FIG. 6 is a view for predicting a blood sugar state of a user based on a pre-stored blood sugar profile in an electronic apparatus according to an embodiment of the present disclosure;

FIGS. 7A and 7B are first views for providing a blood sugar care service based on activity information of a user in an electronic apparatus according to various embodiments of the present disclosure;

FIGS. 8A and 8B are second views for providing a blood sugar care service based on activity information of a user in an electronic apparatus according to various embodiments of the present disclosure;

FIG. 9 is a flowchart of a method for providing a blood sugar care service in an electronic apparatus according to an embodiment of the present disclosure; and

FIG. 10 is a flowchart for setting a target profile in an electronic apparatus according to an embodiment of the present disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Further, like reference numerals indicate like components that perform substantially the same functions throughout the specification. For the sake of explanation and understanding, different embodiments are described with reference to like reference numerals. That is, even if all the components in the plurality of drawings have like reference numerals, it does not mean that the plurality of drawings refer to only one embodiment.

Further, the terms including numerical expressions such as a first, a second and the like may be used to explain various components, but there is no limitation thereto. These terms are used only for the purpose of differentiating one component from another, without limitation thereto. For example, a numerical expression combined with a component should not limit the order of use or order of arrangement of the component. When necessary, the numerical expressions may be exchanged between components.

In this specification, terms such as “include” and “have/has” should be construed as designating that there are such characteristics, numbers, operations, elements, components or a combination thereof in the specification, not to exclude the existence or possibility of adding one or more of other characteristics, numbers, operations, elements, components or a combination thereof.

In the embodiments of the present disclosure, terms such as “module,” “unit,” and “part” and the like are terms used to indicate components that perform at least one function and operation, and these components may be realized in hardware, software or in combination thereof. Further, except for when each of a plurality of “modules,” “units,” “parts” and the like needs to be realized in an individual hardware, the components may be integrated in at least one module or chip and be realized in at least one processor (not illustrated).

Further, in the embodiments of the present disclosure, when it is described that a portion is connected to another portion, the portion may be either connected directly to the other portion, or connected indirectly via another medium. Further, when it is described that a portion includes another component, it does not exclude the possibility of including other components, that is, the portion may further include other components besides the described component.

Hereinafter, various embodiments of the present disclosure will be explained with reference to the drawings attached.

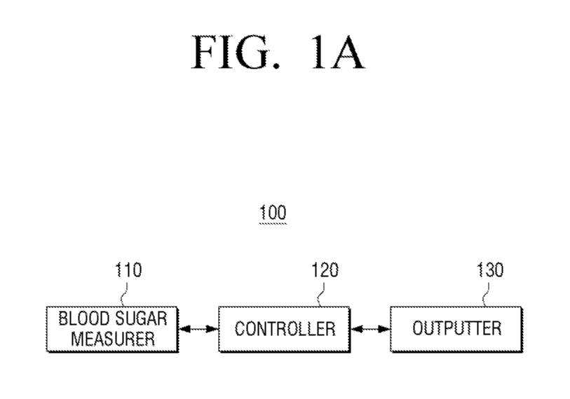

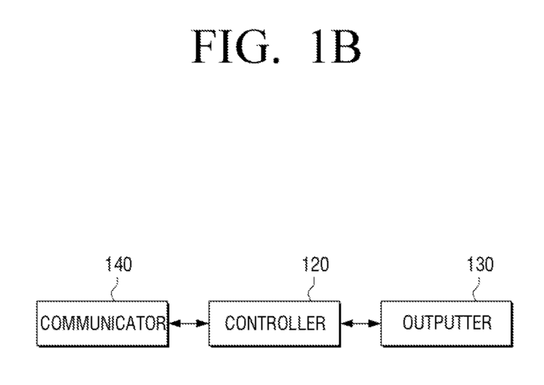

FIG. 1A is a schematic block diagram of an electronic apparatus that measures and cares a user's blood sugar according to an embodiment of the present disclosure, and FIG. 1B is a schematic block diagram of an electronic apparatus that cares a user's blood sugar according to an embodiment of the present disclosure.

Referring to FIG. 1A, the electronic apparatus 100 is an apparatus configured to provide a care service regarding a blood sugar state of a user. It may be a blood sugar care apparatus (not illustrated) that is attached to the user's body to measure the user's blood sugar and provide the user's blood sugar care state based on the measured blood sugar information.

Further, referring to FIG. 1B, the electronic apparatus 100 may be an apparatus configured to receive the blood sugar information measured by a blood sugar measurement apparatus (not illustrated) attached to the user's body and provide the user's blood sugar care state based on the received blood sugar information.

Such an electronic apparatus 100 may be a display apparatus such as, for example, a smart phone, or a wearable device such as smart watch, smart band, smart glass (AR) etc. In the case where such an electronic apparatus 100 is a blood sugar care apparatus (not illustrated) that is attached to the user's body to measure the user's blood sugar and provide a blood sugar care state, the electronic apparatus 100 includes a blood sugar measurer 110, a controller 120 and a controller 130, as illustrated in FIG. 1A.

The blood sugar measurer 110 is inserted into the user's body to regularly measure the user's blood sugar and output the same to the controller 120. In some embodiments, the blood sugar measurer 110 may measure the user's blood sugar in minute units, and output the user's blood sugar measured in minute units to the controller 120.

The controller (i.e., processor) 120 controls the overall operations of a plurality of components of the electronic apparatus 100. For example, the controller 120 obtains blood sugar information on the user's blood sugar regularly measured and output by the blood sugar measurer 110, in predetermined time units. Thereafter, the controller 120 generates a blood sugar profile based on the user's blood sugar information obtained in the predetermined time units. In some embodiments, the controller 110 may generate the blood sugar profile based on the blood sugar information obtained for 24 hours.

When the blood sugar profile is generated as aforementioned, the controller 120 compares a currently generated blood sugar profile with a previously generated blood sugar profile, and sets the blood sugar profile proximate to a normal blood sugar range as a target profile.

Here, the normal blood sugar range may be a blood sugar range section that is a medical standard. Further, of the two blood sugar profiles, the blood sugar profile proximate to the normal blood sugar range may be the blood sugar profile that belongs to the normal blood sugar range or is close to the normal blood sugar range. Therefore, of the currently generated blood sugar profile and the previously generated blood sugar profile, the controller 120 may set the blood sugar profile closer to the normal blood sugar range as the target profile. Meanwhile, after the target profile is set, the controller 120 may generate a blood sugar profile based on the blood sugar information measured by the blood sugar measurer 110. In this case, the controller 120 may compare the blood sugar profile set as the target profile and the currently generated blood sugar profile, and update the predetermined target profile with the blood sugar profile proximate to the normal blood sugar range, of the two blood sugar profiles.

That is, of the blood sugar profile set as the target profile and the currently generated blood sugar profile, if the currently generated blood sugar profile is proximate to the normal blood sugar range, the controller 120 updates the currently generated profile as the target profile. Meanwhile, of the blood sugar profile set as the target profile and the currently generated blood sugar profile, if the blood sugar profile predetermined as the target profile is proximate to the normal blood sugar range, the controller 120 may maintain the predetermined target profile without performing an additional update regarding the predetermined target profile.

When such a target profile is set or the predetermined target profile is updated by a new blood sugar profile, the controller 120 generates blood sugar state information of the user based on the target profile, and controls the outputter 130 to provide the generated blood sugar state information. Accordingly, the outputter 130 may provide the blood sugar state information of the user generated based on the target profile as one of video and audio.

Meanwhile, the controller 120 may match the blood sugar information measured of the user, the blood sugar profile generated based on the blood sugar information and various information to one another, and store the same in a storage 190 to be explained later on. For example, the storage 190 may match at least one of the blood sugar information measured of the user, blood sugar profile generated based on the blood sugar information, a blood sugar care evaluation value computed from the blood sugar profile, situation information including at least one of the day of the week, time, and user location of when the blood sugar of the user was measured, activity information including at least one of information on food that the user took at the time the blood sugar of the user was measured and exercise information, and body information such as the heart beat and respiration at the time the user's blood sugar was measured, and store the same.

Meanwhile, in the case where the electronic apparatus 100 is an apparatus that provides the blood sugar care state of the user based on the blood sugar information received through the blood sugar measurer (not illustrated) attached to the user's body, as illustrated in FIG. 1B, the electronic apparatus may include a communicator 140, a controller 120 and an outputter 130.

For example, the communicator 140 performs data communication with the blood sugar measurement apparatus (not illustrated) that is attached to the user's body and that regularly measures the user's blood sugar, and receives the measured blood sugar information from the blood sugar measurement apparatus (not illustrated).

However, the present disclosure is not limited thereto, and the blood sugar measurement apparatus (not illustrated) that measures the user's blood sugar may transmit the regularly measured blood information of the user to a blood sugar care server (not illustrated) that cares the user's blood sugar. In this case, the communicator 140 may receive the user's blood information through the blood sugar care server (not illustrated).

When the user's blood sugar information is received from such a blood sugar measurement apparatus (not illustrated) or such a blood sugar care server (not illustrated) through the communicator 140, as aforementioned, the controller 120 may generate the blood sugar profile based on the received user's blood information, and compare the generated blood sugar profile with the previously generated blood sugar profile to set the blood sugar profile proximate to the normal blood sugar range as the target profile.

Meanwhile, the controller 120 may set one of the blood sugar profile generated based on the blood sugar information measured of the user and the pre-generated blood sugar profile as the target profile through the following embodiments.

According to an embodiment, the controller 120 may compute a blood sugar care evaluation value corresponding to each of the previously generated blood sugar profile (hereinafter referred to as a first blood sugar profile) and the currently generated profile (hereinafter referred to as a second profile) according to a predetermined condition, and set one of the first and second blood sugar profiles as the target profile based on the computed blood sugar care evaluation value.

For example, when the blood sugar care evaluation value corresponding to each of the first and second blood sugar profile is computed, the controller 120 determines the blood sugar profile having the highest value, of the computed blood sugar care evaluation values, as the blood sugar profile proximate to the predetermined normal blood sugar range, and sets the corresponding blood sugar profile as the target profile.

That is, the controller 120 may determine that the blood sugar care evaluation value having the highest value, of the blood sugar care evaluation values corresponding to a first and a second blood sugar profile, as the blood sugar profile that belongs to the normal blood sugar range or that is proximate to the normal blood sugar range, and set the corresponding blood sugar profile as the target profile.

Meanwhile, the controller 120 may compute the blood sugar care evaluation value corresponding to each of the first and the second blood sugar profile according to a predetermined condition as follows.

For example, the controller 120 may compute the blood sugar case evaluation value corresponding to each of the first and the second blood sugar profile based on at least one of an extent the blood sugar profile is included in the normal blood sugar range, a difference between a maximum blood sugar value and a minimum blood sugar value based on the plurality of blood sugar information included in the blood sugar profile, and a period of time the blood sugar information is maintained outside the normal blood sugar range based on the plurality of blood sugar information included in the blood sugar profile.

For example, a different weighted value may be applied to each of a first condition for determining the extent the blood sugar profile is included in the normal blood sugar range, a second condition for determining the difference between the maximum blood sugar value and the minimum blood sugar value based on the plurality of blood sugar information included in the blood sugar profile, and a third condition for determining the period of time the blood sugar information is maintained outside the normal blood sugar range based on the plurality of blood sugar information included in the blood sugar profile.

For example, based on a maximum of 100 points, the weighted value of 50% may be applied to the first condition, the weighted value of 30% may be applied to the second condition, and the weighted value of 20% may be applied to the third condition. In this case, the controller 120 may gather the scores according to the first to the third conditions and compute the blood sugar care evaluation value of the blood sugar profile.

That is, the controller 120 computes the score of the section where the distribution value % to which the blood sugar profile exists within the normal blood sugar range belongs as the score according to the first condition with reference to the scores by a predetermined distribution section based on a maximum of 50 points.

Further, the controller 120 computes the score of the section where the difference value between the blood sugar information having the maximum blood sugar value and the blood sugar information having the minimum blood sugar of the plurality of blood sugar information included in the blood sugar profile belongs to as the score according to the second condition, with reference to the scores by a predetermined blood sugar unit (mg/dL) section based on a maximum of 30 points.

Further, the controller 120 computes the score of the section where the period of time the plurality of blood sugar information included in the blood sugar profile is continuously maintained outside the normal blood sugar range belongs to as the scores according to the third condition with reference the scores by a predetermined section of time units based on a maximum of 20 points.

For example, 60% of the first blood sugar profile may exist within the normal blood sugar range, the difference between the maximum and the minimum blood sugar value may be 120 mg/dL, and the blood sugar information measured for 2 hours (12:00˜14:00) may exist outside the normal blood sugar range.

Further, 80% of the second blood sugar profile may exist within the normal blood sugar range, the difference between the maximum and the minimum blood sugar value may be 120 mg/dL, and the blood sugar information measured for 3 hours 10:00˜11:00, 13:00˜14:00, 19:00˜20:00 may exist outside the normal blood sugar range.

In this case, the blood sugar care evaluation value of the second blood sugar profile may be a higher value than the blood sugar care evaluation value of the first blood sugar profile. Therefore, the controller 120 may determine that the second blood sugar profile having a higher blood sugar care evaluation value than the first blood sugar profile is proximate to the normal blood sugar range, and set the second blood sugar profile as the target profile.

Meanwhile, the weighted values according to the first to the third condition may be predetermined as in the aforementioned example, set by the user or set by a medical specialist of the corresponding technical field.

In the case where the weighted values according to the first to the third condition are set by the medical specialist, the weighted values according to the first to the third condition may be set differently depending on the body state of the user.

For example, for a user suffering from a heart disease, the medical specialist may set the weighted values according to the first to the third condition of type A, and for a user suffering from diabetes, the medical specialist may set the weighted values according to the first to the third condition of type B.

Meanwhile, the controller 120 may update the aforementioned weighted values according to the first to the third conditions based on at least one of the situation information, activity information and body information matched to the plurality of blood sugar profiles and each blood sugar profile pre-stored. Thereafter, the controller 120 may compute the blood sugar care evaluation value regarding the user's blood sugar profile based on updated the weighted values according to the first to the third condition.

However, the present disclosure is not limited thereto, and as aforementioned, in the case where the weighted values according to the first to the third conditions are set by the medical specialist, the weighted values according to the first to the third condition set by the medical specialist may be stored in an external server (not illustrated) that manages the blood sugar profiles of the plurality of users. In this case, the controller 120 transmits living pattern information including age, gender, occupation, medical history and the like of the user input by the user to the external server (not illustrated) through the communicator 140. Accordingly, the external server (not illustrated) transmits one of the information on the weighted values according to the plurality of the first to the third condition pre-stored based on the user's living pattern information received from the electronic apparatus 100 to the electronic apparatus 100. Accordingly, the external server (not illustrated) transmits one of the information on the weighted value settings according to the plurality of the first to the third condition pre-stored based on the user's living pattern received from the electronic apparatus 100 to the electronic apparatus 100. Accordingly, the controller 120 may compute the blood sugar care evaluation value regarding the blood sugar profile based on the information on the weighted value settings according to the first to the third condition received from the external server (not illustrated). After the target profile is set, when the blood sugar profile is generated based on the plurality of blood sugar information obtained for a predetermined period of time, the controller 120 computes the blood sugar evaluation value of the currently generated blood sugar profile as aforementioned. Thereafter, the controller 120 may compare the blood sugar care evaluation value computed from the currently generated blood sugar profile and the blood sugar evaluation value pre-computed regarding the blood sugar profile set as the target profile, and re-set the blood sugar profile having a higher value, of the two evaluation values, as the target profile.

According to another embodiment, as aforementioned, the controller 120 may set the blood sugar profile suitable to the current situation as the target profile with reference to the situation information, activity information and body information matched to each of the plurality of blood sugar profiles pre-stored in the storage 190.

For example, in the case of a user having different living patterns for different days of the week, the controller 120 may set the blood sugar profile by the day of the week as the target profile based on the situation information of the user pre-stored in the storage 190.

In more detail, a user who goes to work may have different eating habits for weekdays and weekends. That is, in the case of weekdays, the user who goes to work may eat at certain hours but eat at irregular hours during weekends.

In this case, based on the situation information pre-stored in the storage 190, the controller 120 may set the blood sugar profile proximate to the normal blood sugar range, of the blood sugar profiles of weekdays, as the target profile for weekdays, and set the blood sugar profile proximate to the normal blood sugar range, of the blood sugar profiles of weekends, as the target profile for weekends.

However, the present disclosure is not limited thereto, and thus the controller 120 may set the blood sugar profile by day of the week as the target profile based on the pre-stored situation information.

Meanwhile, the controller 120 may change the target profile set according to the living pattern by the day of week of the user.

For example, in the case where the user who goes to work did not go to work due to holiday or a weekday is a holiday, the user's living pattern may be different from a normal weekday living pattern.

In this case, the controller 120 compares location information of where the user is located in weekdays and location information sensed through a sensor 180 to be explained hereinafter based on the situation information pre-stored in the storage 190 and determines whether or not the user is in a similar location.

If the location information sensed through the sensor 180 is different from the location information of where the user is located in weekdays as a result of the determination, the controller 120 determines that the user's living pattern is different from the normal living pattern of weekdays.

If it is determined that the user's living pattern is different from the normal living pattern of weekdays, the controller 120 may change the predetermined target profile of weekends to the target profile of weekdays.

In another example, the controller 120 may set the blood sugar profile related to the user's current location as the target profile based on the user's situation information pre-stored in the storage 190.

For example, the user may be eating in a restaurant or exercising in a training center. In this case, the controller 120 may obtain location information on the restaurant where the user is eating or where the training center is located through the sensor 180. When such a current location information of the user is obtained, the controller 120 determines whether or not the location information related to the current location information of the user is stored based on the situation information of the user pre-stored in the storage 190. If the location information related to the current location information of the user is stored as a result of determination, the controller 120 may set the blood sugar profile matched to the location information related to the current location information of the user, of the pre-stored plurality of blood sugar profiles, as the target profile.

In another example, the controller 120 may set the blood sugar profile that the user selected from the plurality of blood sugar profiles pre-stored in the storage 190 as the target profile.

In another example, the controller 120 may receive the blood sugar profile of another user related to the predetermined condition from the external server (not illustrated) that manages the blood sugar profiles of a plurality of users, and set the blood sugar profile of the other user received as the target profile.

For example, the controller 120 transmits the living pattern information that includes the age, gender, occupation and medical history of the user input by the user to the external server (not illustrated). Accordingly, the external server (not illustrated) obtains the blood sugar profile of another user having a similar living pattern as the corresponding user, of the blood sugar profiles of a plurality of users pre-stored based on the living pattern information of the user received from the electronic apparatus 100, and transmits the blood sugar profile proximate to the normal blood sugar range, of the obtained blood sugar profiles, to the electronic apparatus 100. Accordingly, the controller 120 may set the blood sugar profile of the other user received from the external server (not illustrated) as the target profile.

Meanwhile, the controller 120 may set the target profile through the aforementioned embodiments, and then change the predetermined target profile based on the blood sugar information measured of the user regularly.

For example, after the target profile is set, the controller 120 obtains the blood sugar information measured of the user regularly for a certain period of time, and then generates the blood sugar profile based on the blood sugar information obtained for a certain period of time. Thereafter, the controller 120 determines whether or not there is a blood sugar profile, of the plurality of pre-stored blood sugar profiles, that is similar to the currently generated blood sugar profile. If there is a blood sugar profile pre-stored that is similar to the currently generated blood sugar profile, the controller 120 may automatically change the blood sugar profile similar to the currently generated blood sugar profile as the target profile.

However, the present disclosure is not limited thereto, and the controller 120 may output a notifying message for changing the target profile through the outputter 130, and when a user command is input in response, the controller 120 may change the blood sugar profile similar to the currently generated blood sugar profile as the target profile.

For example, the controller 120 determines the current state of the user based on the sense information sensed through the sensor 180. That is, the sensor 120 may determine whether the user is driving, walking or running based on the sense information sensed through the sensor 180, and control the outputter 130 to output a notifying message for changing the target profile in at least one of an image, video or alarm based on the determined current state of the user. Accordingly, the outputter 130 outputs a notifying message for changing the target profile in at least one of the image, voice and alarm, and if a user command is input after the notifying message is output, the controller 120 may change the blood sugar profile similar to the currently generated blood sugar profile as the target profile.

After the target profile is set through such various embodiments, when the blood sugar information is obtained, the controller 120 may provide the blood sugar state of the user through embodiments as follows.

According to an embodiment, after the target profile is set, when the blood sugar information measured of the user is obtained, the controller 120 may compare the blood sugar information of the time point when the current blood sugar information was obtained and the current obtained blood sugar information, of the plurality of blood sugar information constituting the target profile, and provide the current blood sugar state of the user.

According to another embodiment, after the target profile is set, when the blood sugar profile is generated based on the blood sugar information measured of the user, the controller 120 may analyze the currently generated blood sugar profile based on at least one of the target profile and the normal blood sugar range, and provide the blood sugar care state of the user by period.

For example, the controller 120 may provide the blood sugar care state of the user by period through the embodiments as follows.

According to an embodiment, in order to evaluate the blood sugar care state of the user, the controller 120 may provide the blood sugar care state of the user by period based on an evaluation grade section where an extent (distribution)(%) where the currently generated blood sugar profile belongs to the normal blood sugar range or an error extent (%) between the currently generated blood sugar profile and the target profile belongs to, of the plurality of evaluation grade sections.

For example, the currently generated blood sugar profile may belong to the normal blood sugar range, and the error range between the currently generated blood sugar profile and the predetermined target profile may be less than 20%. In this case, the controller 120 may determine that the blood sugar care of the user belongs to a first evaluation grade, which indicates that the blood sugar care of the user is in a good state, and provide the blood sugar care state of the user by period accordingly.

Meanwhile, the currently generated blood sugar profile may not belong to the normal blood sugar range, and the error range between the currently generated profile and the predetermined target profile may be less than 50%. In this case, the controller 120 may determine that it belongs to a second evaluation indicating a state where the user needs blood sugar care, and provide the blood sugar care state of the user by period accordingly.

Meanwhile, the currently generated blood sugar profile may be lower or higher than the normal blood sugar value, which represents a low blood sugar or a high blood sugar state, and the error range between the currently generated blood sugar profile and the predetermined target profile may be 50% or above. In this case, the controller 120 may determine that that belongs to a third evaluation grade indicating that the user's blood sugar is not being taken care of, and provide the blood sugar care state of the user by period accordingly.

According to an additional aspect of the present disclosure, the controller 120 may generate a blood sugar history profile based on the blood sugar information included in each of the plurality of blood sugar profiles generated in a predetermined time unit.

When such a history profile is generated, the controller 120 determines the section that exists outside the normal blood sugar range based on the generated blood sugar history profile. Thereafter, the controller 120 may predict a period where the user's blood sugar will go outside the normal blood sugar range based on the section determined as existing outside the normal blood sugar range.

For example, based on the normal blood sugar range, the controller 120 may predict that blood sugar that is lower than the normal blood sugar range will be measured in a first to a second section (06:00˜07:00) through a blood sugar history profile generated from a plurality of blood sugar profiles generated in a predetermined time unit. When such a blood sugar state of the user is predicted, the controller 120 may provide the blood sugar state information predicted at a previous time than the time of the section was predicted to show a blood sugar lower than the normal blood sugar range.

Meanwhile, as aforementioned, the controller 120 may provide information on the blood sugar that may approach the normal blood sugar range together with the blood sugar rate information of the user predicted at a previous time than the section predicted to go outside the normal blood sugar range.

For example, when the section predicted to go outside the normal blood sugar range is determined from the blood sugar history profile generated from the plurality of blood sugar profiles, the controller 120 may provide the information on the blood sugar care that may approach the normal blood sugar range with reference to at least one of the situation information and activity information matching the blood sugar information of the corresponding section.

For example, the controller 120 may provide the information on the blood sugar care including diet information that will allow the section predicted to go outside the normal blood sugar range to approach the normal blood sugar range based on the information on food taken by the user included in the activity information matching the blood sugar information belonging to the normal blood sugar range.

According to an additional aspect of the present disclosure, when activity information is input by the user, the controller 120 stores the blood sugar information measured at the time point where the corresponding activity information was generated to the storage 190 to be explained hereinafter. Here, the activity information may include at least one of food information taken by the user and the exercise information performed by the user. Further, the time point when the activity information was generated may be the time the user ate food or the time the user exercised.

Here, the food information includes at least one of the time the user ate the food, the type of the food, ingredients of the food and amount of the food. The exercise information may include at least one of the type of the exercise, the time point at which the exercise started, and body changes (for example, changes in heart beat and body weight).

Therefore, when such activity information is input, the controller 120 may store the type of the food that the user ate, the time the user ate the food, and the blood sugar information measured at the time the user ate the food, or store the type of the exercise performed by the user, the duration time of the exercise, and the blood sugar information measured during the duration of the exercise.

Therefore, after the target profile is set, when the blood sugar information measured of the user is obtained, the controller 120 may provide the pre-stored activity information based on the blood sugar information at the point where the pre-obtained blood sugar information is located.

According to an additional aspect of the present disclosure, the controller 120 transmits the blood sugar profile generated in the predetermined time unit to the external server (not illustrated) through the communicator 140 that will be explained hereinafter. Accordingly, the external server (not illustrated) generates ranking information of the blood sugar care group to which the corresponding user belongs based on the blood sugar profile of the user received from the electronic apparatus 100.

For example, when the blood sugar profile of the user is received from the electronic apparatus 100, the external server (not illustrated) compares and analyzes the blood sugar profile of the corresponding user and the blood sugar profile of the users within the blood sugar care group to which the corresponding user belongs, determines the ranking in the order of blood sugar profiles most proximate to the predetermined normal blood sugar section, and transmits the ranking information of the corresponding user to the electronic apparatus 100. Accordingly, the controller 120 may provide the ranking information of the blood sugar care group received from the external server (not illustrated).

So far, operations of the electronic apparatus 100 according to embodiments of the present disclosure to provide blood sugar care service of users were explained. Hereinafter, configurations of the electronic apparatus 100 configured to provide blood sugar care service of users will be explained.

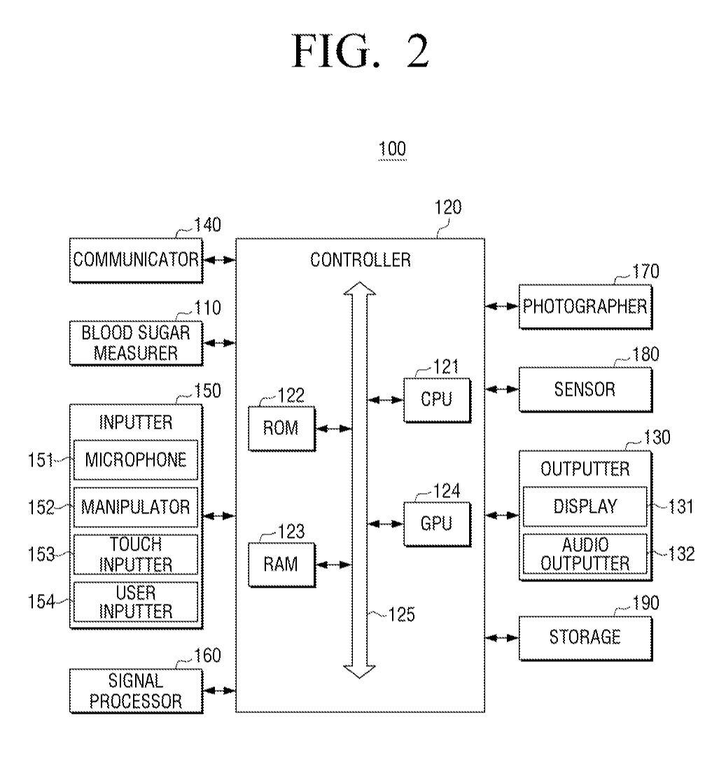

As aforementioned, in the case where the electronic apparatus 100 is realized as a display apparatus such as smart phone providing blood sugar state of the user based on the blood sugar information received through the blood sugar measurement apparatus (not illustrated) attached to the user's body, the electronic apparatus 100 may further include the configuration as in FIG. 2.

FIG. 2 is a block diagram of the electronic apparatus according to an embodiment of the present disclosure.

Referring to FIG. 2, the electronic apparatus 100 may be a blood sugar care apparatus (not illustrated) attached to a user's body to measure the blood sugar of the user and provide the blood sugar state of the user based on the measured blood sugar information, or an apparatus that receives the blood sugar information measured through the blood sugar measuring apparatus (not illustrated) attached to the user's body and provides the blood sugar care state of the user based on the received blood sugar information.

In this case, the electronic apparatus 100 may include a blood sugar measurer 110, a controller 120 and an outputter 130, as illustrated in the aforementioned FIG. 1A, or include a communicator 140, a controller 120 and an outputter 130, as illustrated in the aforementioned FIG. 1B.

Such an electronic apparatus 100 may further include an inputter 150, a signal processor 160, a photographer 170 a sensor 180 and a storage 190, as further illustrated in FIG. 2.

Prior to explaining the additional configurations, it is to be noted that the aforementioned outputter 130 includes a display 131 and an audio outputter 132, as illustrated in FIG. 2.

The display 131 may display, on a screen, a contents image received from a contents server (not illustrated) and an external source apparatus (not illustrated), or pre-stored in the storage 190. For example, the display 131 may display on the screen the blood sugar state information of the user based on the aforementioned target profile.

The audio outputter 132 may output, through a speaker (not illustrated), a contents audio received from one of the contents server (not illustrated) and the external source apparatus (not illustrated) or pre-stored in the storage 190, in an audible form. For example, the audio outputter 132 may output the blood sugar state of the user in an audio form based on the aforementioned target profile.

Meanwhile, the display 131, configured to display the content image or the blood sugar state information of the user, may be realized as a liquid crystal display (LCD), an organic light emitting display (OLED), or a plasma display panel (PDP). For example, the display 131 may be realized in a touch screen form forming a mutual layer structure together with a touch inputter 153 included in an inputter 150 that will be explained later on.

The communicator 140 performs data communication with the blood sugar measurement apparatus (not illustrated) attached to the user's body to receive blood sugar information regularly measured by the blood sugar measuring apparatus (not illustrated). Not only that, the communicator 140 may perform data communication with a plurality of pre-registered peripheral devices (not illustrated), contents server, web server (not illustrated) and receive contents.

Such a communicator 140 may include a wireless communication module such as short distance communication module and wireless local area network (LAN) module, and a wired communication module such as high-definition multimedia interface (HDMI), universal serial bus (USB), Institute of Electrical and Electronics Engineers (IEEE) 1394, etc.

The short distance communication module is a communication module for performing wireless communication with the plurality of pre-registered peripheral apparatuses (not illustrated), and may include at least one of an infrared data association (IrDA) module, near field communication (NFC) module, Wi-Fi module and ZigBee module.

Further, the wireless LAN module is a module such as IEEE and the like configured to be connected to an external network according to a wireless communication protocol, and may perform data communication with web server (not illustrated), contents server (not illustrated) and the like.

Besides the aforementioned, the wireless communication module may further include a mobile communication module that may be connected to a mobile communication network according to various mobile communication standards such as 3rd generation (3G), 3G partnership project (3GPP), long term evolution (LTE) to perform communication.

As such, the communicator 140 may be realized by various aforementioned short distance communication methods, and may adopt other communication technologies not mentioned in the present specification when necessary.

Meanwhile, a wired communication module is a configuration that provides an interface with various source apparatuses such as USB 2.0, USB 3.0, HDMI, IEEE 1394 and the like. Such a wired communication module may receive contents data transmitted from an external server (not illustrated) via a wired cable or transmit pre-stored contents data to an external record medium according to a control command of the controller 120. Further, the wired communication module may receive input of power from a power source via a wired cable.

The inputter 150 is an input means for receiving input of various user commands to be transmitted to the controller 120, and includes a microphone 151, a manipulator 152, the touch inputter 153 and a user inputter 154.

The microphone 151 may receive input of a user's voice command, and the manipulator 152 may be realized as a key pad provided with various function keys, number keys, special keys, letter keys and the like. Further, in the case where the aforementioned display 131 is realized in a touch screen form, the touch inputter 153 may be realized as a touch pad forming a mutual layer structure with the display 131. In this case, the touch inputter 153 may receive input of a command to select at least one of various application related icons and a user interface (UI) for execution of an application through the display 131.

The user inputter 154 may receive input of an infrared (IR) signal or radio frequency (RF) signal for controlling operations of the electronic apparatus 100 from at least one peripheral device (not illustrated) such as a remote control apparatus.

The signal processor 160 may be a component for processing image data and audio data of contents received through the communicator 140 or of contents stored in the storage 190. For example, the signal processor 160 may perform various image processing such as decoding, scaling, noise filtering, frame rate conversion and resolution conversion on the image data included in the contents. Further, the signal processor 160 performs various audio signal processing such as decoding, amplifying, noise filtering and the like on the audio data included in the contents, and the signal processed audio data is output in an audible form through the audio outputter 132.

The photographer 170 is for photographing a still image or video according to a user command. The photographer 170 may be realized in plural, including a front camera, rear camera and the like.

The sensor 170 is a sensor that senses brightness of a surrounding area and motion of the display apparatus 100. Such a sensor 180 may include an illuminance sensor, an accelerometer sensor, a terrestrial magnetic sensor, gravity sensor and gyroscope sensor etc.

The illuminance sensor senses brightness of a surrounding area, and the accelerometer sensor is an acceleration sensor that measures acceleration of the electronic apparatus 100 or intensity of impact. Further, the magnetic sensor is a sensor capable of detecting azimuth using the earth's magnetic field, and the gravity sensor is a sensor for detecting in which direction gravity takes effect, which automatically rotates according to the direction in which the user holds the electronic apparatus to sense the direction. Lastly, the gyroscope sensor is a sensor made by applying rotation to an accelerometer sensor of the related art to perceive 6 axis directions to help recognize more detailed and precise operations.

Meanwhile, in the case where the electronic apparatus 100 is a blood sugar care apparatus (not illustrated) that is attached to a user's body to measure the user's blood sugar, and that provides the blood sugar care state of the user based on the measured blood sugar information, the sensor 180 may further include a sensor that senses the body state of the user such as heartbeat sensor, temperature sensor and blood flow measurement sensor etc.

The storage 190 stores execution programs, contents and the like regarding various applications. For example, as mentioned above, the storage 190 stores the blood sugar information regularly measured of the user through the blood sugar measurer 110, and stores the blood sugar profile generated based on the blood sugar information measured for a predetermined period of time and the blood sugar profile set as the target profile.

Here, as mentioned above, the storage 190 may match various information such as the blood sugar information measured of the user, the blood sugar profile generated based on the blood sugar information and other various information to one another and store the same. For example, the storage 190 may match at least one of blood sugar information measured of the user, blood sugar profile generated based on the blood sugar information, blood sugar care evaluation value computed from the blood sugar profile, situation information including at least one of the day of week and time the blood sugar of the user was measured, activity information including at least one of information on food taken by the user and exercise information, and body information such as the heart beat and respiration at a time point the user's blood sugar was measured, and store the same.

Further, the storage 190 may further store an operating program for controlling the operations of the electronic apparatus 100. Here, the operating program may be a program that is read in the storage 190 when the electronic apparatus 100 is turned on, and is compiled to operate each configuration of a user terminal apparatus 100.

Meanwhile, the aforementioned controller 120 may include a central processing unit (CPU) 121, a read only memory (ROM) 122, a random access memory (RAM) 123 and a graphics processing unit (GPU) 124, and the CPU 121, the ROM 122, the RAM 123 and the GPU 124 may be connected to each other via bus 125.

The CPU 121 accesses the storage 190 and uses the operating system (OS) stored in the storage 190 to boot the system. Further, the CPU 121 performs various operations using various programs, contents, data and the like stored in the storage 190.

The ROM 122 stores sets of commands for the system and the like for system booting. When a turn-command is input and power is supplied, the CPU 121 copies the OS stored in the storage 190 and boots the system according to commands stored in the ROM 12. When booting is completed, the CPU 121 copies various programs stored in the storage 190 to the RAM 123, and executes the programs copied in the RAM 123 to perform various operations.

The GPU 124 generates a display screen that includes various objects such as icon, image, text and the like. For example, the GPU 124 computes attribute values such as coordinate values, forms, sizes, colors and the like for displaying each object on a layout of a screen based on a control command received, and generates a display screen of various layouts including the objects.

Such a controller 120 may be combined with each of the aforementioned configurations and be realized in a system-on-a-chip or a system on chip (SOC, SoC) and the like.

Meanwhile, the aforementioned operations of the controller 120 may be performed by the programs stored in the aforementioned storage 190. Here, the storage 190 may be realized as at least one of the ROM 122, the RAM 123 or a memory card (e.g., a secure digital (SD) card, a memory stick, etc.) attachable/detachable to/from the electronic apparatus 100, nonvolatile memory, volatile memory, hard disk drive (HDD) or solid state drive (SSD).

So far, each configuration and operation of the electronic apparatus 100 according to the present disclosure were explained. Hereinafter, operations of providing a blood sugar state of a user based on the blood sugar information measured of the user in the electronic apparatus 100 will be explained.

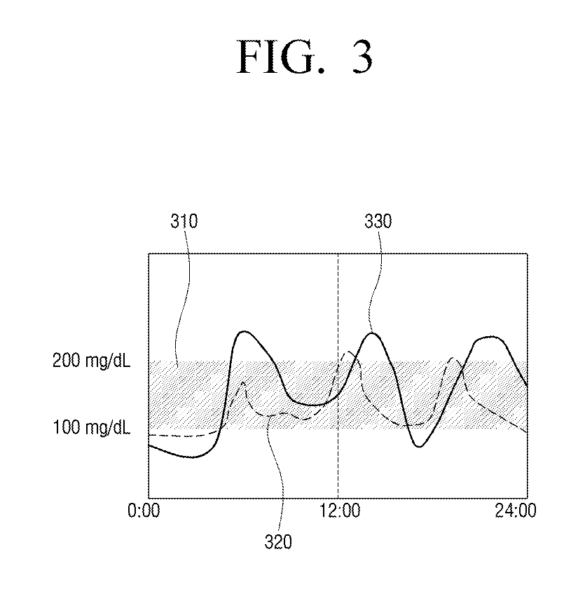

FIG. 3 is a view for setting a target profile in an electronic apparatus according to an embodiment of the present disclosure.

Referring to FIG. 3, the electronic apparatus 100 may receive the blood sugar information regularly measured by the blood measurement apparatus (not illustrated) attached to a user's body. In this case, when the blood sugar information received from the blood sugar measurement (not illustrated) is collected during a predetermined period of time, the electronic apparatus 100 generates a blood sugar profile based on the blood sugar information collected during the predetermined period of time.

For example, the blood sugar measurement apparatus (not illustrated) may measure the user's blood sugar in minute units, and transmit the blood sugar information measured in minute units to the electronic apparatus 100. In this case, the electronic apparatus 100 may generate a blood sugar profile based on the blood sugar information received from the blood sugar measurement apparatus (not illustrated) from 0:00 to 24:00.

When the blood sugar profile is generated based on the blood sugar information collected during a predetermined period of time as aforementioned, the currently generated blood sugar profile 320 and a previously generated blood sugar profile 330 are compared to each other, and of the two blood sugar profiles, the blood sugar profile proximate to a predetermined normal blood sugar section is determined.

That is, the electronic apparatus 100 may compute a blood sugar care evaluation value corresponding to the currently generated blood sugar profile and the previously generated blood sugar profile 320, 330 based on a predetermined condition, and compare each blood sugar care evaluation value computed to determine the blood sugar profile having a higher evaluation value as the blood sugar profile proximate to the predetermined normal blood sugar section 310.

A method for computing the blood sugar care evaluation value regarding the blood sugar profile was already explained through FIGS. 1A and 1B, and thus hereinafter, a detailed explanation will be omitted.

As illustrated, of the blood sugar profiles 320, 330 generated currently and previously, the blood sugar care evaluation value of the currently generated blood sugar profile 320 may have a higher value than the blood sugar care evaluation value of the previously generated blood sugar profile 330. In this case, the electronic apparatus 100 determines that the currently generated blood sugar profile 320 is more proximate to the predetermined normal blood sugar section 310 that the previously generated blood sugar profile 330.

Therefore, the electronic apparatus 100 may set the blood sugar profile 320 determined to be proximate to the predetermined normal blood sugar section 310 as the target profile.

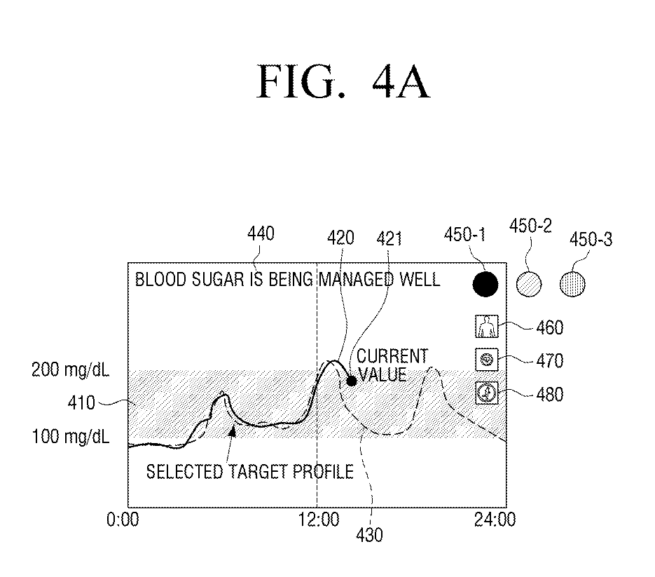

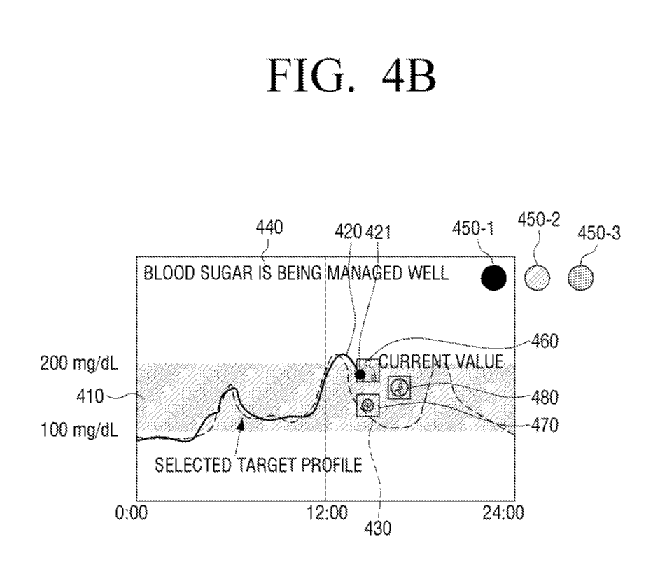

FIG. 4A is a first view providing a blood sugar state of the user based on a predetermined target profile in an electronic apparatus according to an embodiment of the present disclosure, and FIG. 4B is a second view providing a blood sugar state of the user based on a predetermined target profile in an electronic apparatus according to an embodiment of the present disclosure.

Referring to FIG. 4A, a target profile 430 proximate to the predetermined normal blood sugar section 410 may be set, and the electronic apparatus 100 may receive the blood sugar information regularly measured by the blood sugar measurement apparatus (not illustrated) attached to the user's body.

As aforementioned, when the blood sugar information regularly measured by the blood sugar measurement apparatus (not illustrated) is received in a state where the target profile 430 is set, the electronic apparatus 100 overlaps the blood sugar profile 420 generated based on the blood sugar information received from the blood sugar measurement apparatus (not illustrated) to the target profile 430 and displays the same. As aforementioned, as the electronic apparatus provides information on the blood sugar state representing changes in the blood sugar of the user up to the present point based on the target profile 430, the user may visually perceive whether or not the blood sugar state up the present point is being maintained with reference to the predetermined target profile.

For example, when blood sugar information measured from 0:00 to 14:00 is received from the blood sugar measurement apparatus (not illustrated), the electronic apparatus 100 may overlap the blood sugar profile 420 generated based on the blood sugar information measured from 0:00 to 14:00 on the predetermined target profile 430 and display the same.

Here, the electronic apparatus 100 may compare and analyze the blood sugar profile 420 generated from the blood sugar information from 0:00 to 14:00 based on at least one of the normal blood sugar section 410 and the predetermined target profile 430, and display the analyzed result in the form of a UI.

For example, the error range of measuring similarity between the blood sugar profile 420 generated from the blood sugar information measured from 0:00 to 14:00 and the predetermined target profile 430 may be less than 20%. In this case, the electronic apparatus 100 may display a text image 440 stating ‘blood sugar is being cared well’ and a first identification image 450-1 indicating that blood sugar is being cared well on one side of the screen.

Accordingly, the user may determine the state of blood sugar care of the user up to the present point with reference to the text image 440 or the identification image 450-1 displayed on the screen of the electronic apparatus 100.

If the blood sugar profile 430 generated from the blood sugar information measured from 0:00 to 14:00 exists outside the predetermined normal blood sugar range, and the error range of measuring similarity between the blood sugar profile 430 and the predetermined target profile is less than 50%, the electronic apparatus 100 may provide a text image 440 stating that ‘blood sugar needs to be taken care of’ and a second identification image 450-2 indicating that blood sugar care is necessary.

The blood sugar profile 420 generated from the blood sugar information measured from 0:00 to 14:00 may be lower or higher than an abnormal blood sugar value indicating a low blood sugar or high blood sugar state, and the error range between the currently generated blood sugar profile 420 and the predetermined target profile 430 may be 50% or above. In this case, the electronic apparatus 100 may provide a text image 440 stating ‘blood sugar is not being taken care of’ and a third identification image 450-3 indicating that the state of blood sugar is very dangerous.

In addition, together with the blood sugar profile 420 generated based on the blood sugar information measured from 0:00 to 1400, as illustrated, the electronic apparatus 100 may display a first to a third icon 460 to 480 indicating an activity state of the user during a certain period on one area of the screen.

Herein, the first icon 460 is an icon for providing the activity information including at least one of the information on the food taken by the user and exercise information of the user during the period where the user's blood sugar information was measured. Further, the second icon 470 is an icon for providing the activity information including at least one of the information on the food taken by the user and the exercise information of the user during the period where the blood sugar profile set as the target profile 430 was generated. Further, the third icon 480 is an icon for providing the activity information that is helpful for the blood sugar care.

Meanwhile, the aforementioned first to third icons 460 to 480 may each be displayed on an area related to the first to the third icon 460 to 480, respectively, as illustrated in FIG. 4B. For example, the first icon 460 may be displayed to overlap the blood sugar profile 420 generated during the period the blood sugar information of the user was measured, the second icon 470 may be displayed to overlap the target profile 430, and the third icon 480 may be displayed not to overlap the blood sugar profile 420 and the target profile 430.

For example, while the blood sugar information of the user measured from 0:00 to 14:00 is being received from the blood sugar measurement apparatus (not illustrated), the electronic apparatus 100 may receive input of the information on the food taken by the user or the exercise information of the user from the user or from the external server (not illustrated).

Here, the external server (not illustrated) may be a server that performs data communication with a health device to receive exercise information of the user such as amount of exercise, type of exercise, exercise duration and the like, and that transmits the received exercise information to the electronic apparatus 100. Such an external server (not illustrated) may be a personal medical record (PMR)/personal health record (PHR), electronic medical record (EMR), electronic health record (EHR), etc.

When such activity information of the user is input by the user or received from the external server (not illustrated), the electronic apparatus displays the blood sugar profile 420 generated based on the blood sugar information of the user measured from 0:00 to 14:00 and the first icon 460 indicating the activity information of the user input or received.

Further, the electronic apparatus displays the second icon 470 indicating the activity information of the user pre-stored regarding the blood sugar profile 430 set as the target profile and the third icon 480 for providing the activity information that is helpful to blood sugar care of the user.

Thereafter, when a user's command to select one of the first to the third icon 460 to 480 displayed on the screen is input, the electronic apparatus 100 may provide the activity information of the user related to the icon corresponding to the user's select command input.

For example, while the blood sugar profile 420 of the user is being generated based on the blood sugar information measured from 0:00 to 14:00, the activity information of the user regarding breakfast from 08:00 to 08:30, and jogging from 09:00 to 10:00, may be input by the user or received from the external server (not illustrated). Further, regarding the blood sugar profile 430 set as the target profile, the activity information of the user regarding breakfast from 08:00 to 08:30, jogging from 09:00 to 10:00, lunch from 12:00 to 13:00, tennis from 03:00 to 04:00, and dinner from 19:00 to 20:00, may be pre-stored.

Therefore, when a command to select the first icon 460 is input by the user, the electronic apparatus 100 may provide information on food including at least one of the time the user had breakfast, type of food taken during breakfast, and amount of food taken by the user, and exercise information including at least one of the type of exercise of the user, the time the exercise started, the time of duration of the exercise and body changes (for example, heartbeat change and body weight change).

For example, when a command to select the first icon 460 is input, the electronic apparatus 100 may display the activity information (exercise, food intake, etc.) of the user during the period where the blood sugar profile 420 was generated in the form of a graph. Here, the electronic apparatus 100 may display the corresponding graph to overlap the blood sugar profile 420 or convert to a new screen and display the corresponding graph.

Not only that, when a command to select the first icon 460 is input, the electronic apparatus 420 may display the activity information of the user on the blood sugar profile 420 in consideration of the activity time by activity information (exercise, food intake, etc.) of the user recorded during the period the blood sugar profile 420 was being generated.

For example, regarding the blood sugar profile 420, if the user had breakfast from 10:00 to 11:00, it is possible to display food related activity information on a certain section including the time the user had breakfast, of the entire sections of the blood sugar profile 420.

Meanwhile, when a command to select the second icon 470 is input by the user, regarding the blood sugar profile 430 set as the target profile, the electronic apparatus 100 may provide the food information including at least one of the time the user had breakfast, time the user had lunch and dinner, type of food taken by the user during breakfast, ingredient and amount of food taken by the user, and the exercise information including at least one of the type of exercise, starting time of exercise, exercise duration, and body changes (for example, heartbeat changes and body weight changes).

For example, when a command to select the second icon 470 is input, the electronic apparatus 100 may display the activity information of the user (exercise and food intake, etc.) during the period the blood sugar profile set as the target profile 430 was generated in the form of a graph. Here, the electronic apparatus 100 may display the corresponding graph to overlap the target profile 430 or convert to a new screen and display the corresponding graph.

Not only that, when a command to select the second icon 470 is input, the electronic apparatus 100 may display the activity information of the user on the target profile 430 in consideration of the activity time by the activity information (exercise and food intake, etc.) of the user recorded during the period where the blood sugar profile set as the target profile 430 as generated.

For example, regarding the blood sugar profile set as the target profile 430, if the user had breakfast from 08:00 to 08:30, it is possible to display the food related activity information on a certain section including the time of breakfast, of the entire sections.

Meanwhile, when a command to select third icon 480 is input from the user, the electronic apparatus 100 may compare and analyze the blood sugar profile 420 generated based on the blood sugar information measured of the current user and the blood sugar profile 430 set as the target profile and provide activity information that is helpful to the blood sugar care of the user.

When a command to select the third icon 480 is input by the user as aforementioned, the electronic apparatus 100 predicts the state of blood sugar of the user after the present time point 421 where the blood sugar information was measured of the user with reference to the blood sugar profile 430 set as the target profile. As illustrated, the blood sugar of the user after the present time point 421 where the blood sugar information is measured of the user based on the blood sugar profile 430 set as the target profile continuously decreases, and during the section where the user's blood sugar continues to decrease, the activity information of the where the user played tennis in the past during the section where the user's blood sugar continued to decrease may be pre-stored.

Therefore, when a command to select the third icon 480 is input by the user, the electronic apparatus 100 may provide activity information to play less tennis than the past, or recommend to eat more food after playing tennis based on the activity information of the user playing tennis, during the section where the user's blood sugar continues to decrease.

For example, when a command to select the third icon 480 is input, the electronic apparatus 100 may provide the activity information recommending exercise or food intake at the present time point 421 where the blood sugar information is measured of the current user in the blood sugar profile 420 generated based on the blood sugar information measured of the user, in the form of text of image.

Meanwhile, each of the aforementioned first to the third icon 460 to 480 may be changed according to the activity state of the user and be displayed.

When the exercise information and food information of the user are received from a peripheral device communicable with the external server (not illustrated) or the electronic apparatus 100 while the blood sugar information of the user measured from 0:00 to 14:00 is being received from the blood sugar measurement apparatus (not illustrated) as in the aforementioned example, the electronic apparatus 100 may generate an icon for providing the exercise related activity information of the user and an icon for providing food related activity information of the user, independently from each other, and display them on the screen.

Not only that, the electronic apparatus 100 may generate an icon for providing activity information corresponding to each of exercise and food based on the activity information of the user during the period where the blood sugar profile set as the target profile 430 was generated, and display the generated icon on the screen.

Not only that, the electronic apparatus 100 compares and analyzes the blood sugar profile 420 generated based on the blood sugar information measured of the current user the blood sugar profile set as the target profile 430 and predicts the state of blood sugar of the user after the present time point 421 the blood sugar information was measured of the user. Thereafter, the electronic apparatus 100 may generate and display an icon indicating activity information for increasing or reducing blood sugar from the predicted state of blood sugar of the user.

For example, when it is predicted that the blood sugar of the user continued to reduce since the present time point 421 the blood sugar information was measured of the user, the electronic apparatus 100 may generate and display an icon for providing activity information related to food intake.

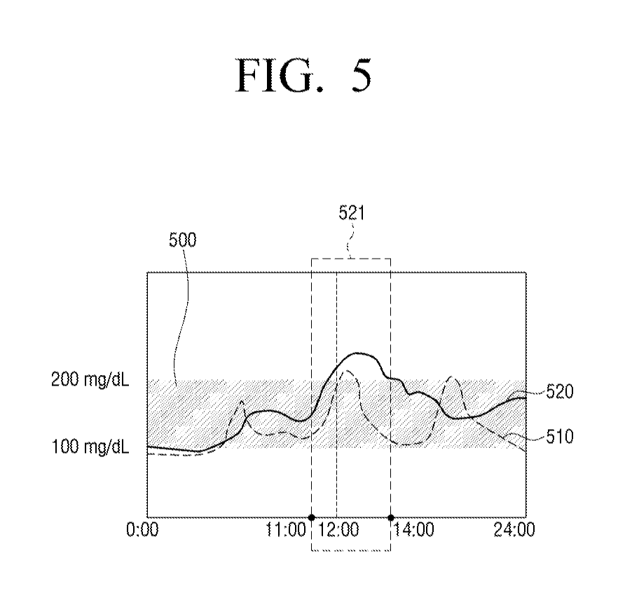

FIG. 5 is a third view providing a state of blood sugar of a user based on a predetermined target profile in an electronic apparatus according to an embodiment of the present disclosure.

Referring to FIG. 5, a predetermined normal blood sugar section 500 and a proximate target profile 510 may be set, and after a target profile 510 is set, the electronic apparatus 100 may generate a blood sugar profile 520 based on the blood sugar information received for a predetermined time unit through the measurement apparatus (not illustrated) attached to the user's body.

When the blood sugar profile 520 is generated with the target profile 510 set as aforementioned, the electronic apparatus 100 may display the blood sugar profile 520 to overlap the target profile 510. Further, the electronic apparatus 100 compares and analyzes the target profile 510 and the blood sugar profile 520.

For example, the electronic apparatus 100 may generate the blood sugar profile 520 based on the blood sugar information received for 24 hours. In this case, the electronic apparatus 100 may compare and analyze the target profile 510 and the blood sugar profile 520 and figure out the changes in the user's state of blood sugar for the 24 hours.

As illustrated, of the plurality of blood sugar information constituting the blood sugar profile 520 with reference to the target profile 510, the blood sugar information 521 measured from 11:00 to 14:00 may exist outside the predetermined normal blood sugar section 500.

In this case, the electronic apparatus 100 may display on the screen at least one of a text image and an identification image indicating that blood sugar was not taken care of between 11:00 and 14:00.

Therefore, the user may not only check the changes in his/her state of blood sugar during the day through the target profile 510 and the blood sugar profile 520 displayed on the screen, but also check during which time of the day his/her blood sugar was not taken care of.

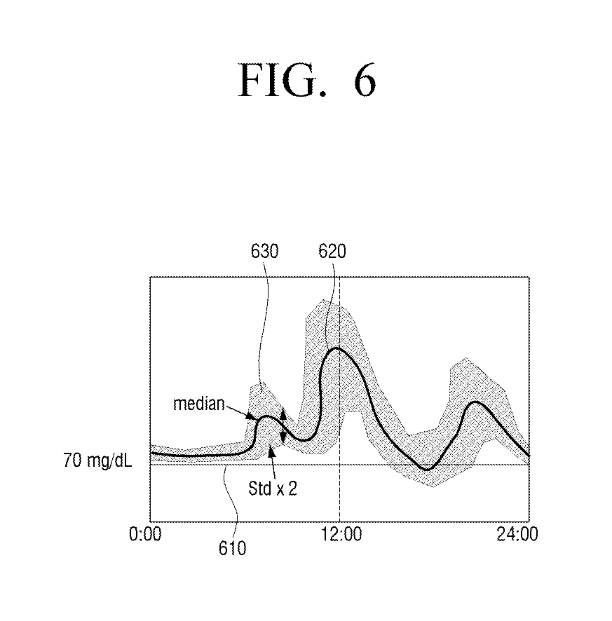

FIG. 6 is a view for predicting and providing a state of blood sugar of a user based on a pre-stored blood sugar profile in an electronic apparatus according to an embodiment of the present disclosure.

Referring to FIG. 6, when at least two or more pre-generated blood sugar profiles are stored, the electronic apparatus 100 may generate a blood sugar history profile 620 using the at least two or more pre-stored blood sugar profiles.

For example, the electronic apparatus 100 may generate and store a blood sugar profile based on the blood sugar information received from the blood sugar measurement apparatus (not illustrated) for 24 hours. When the blood sugar profile generated in a time unit of 24 hours is stored as aforementioned, the electronic apparatus 100 may generate a blood sugar history profile 620 using a plurality of blood sugar profiles stored in units of one week.

According to an embodiment, when a blood sugar profile is stored in units of one week, the electronic apparatus 100 may generate the blood sugar history profile 620 using an average value computed from the blood sugar information measured at times corresponding to each other, of the plurality of blood sugar information constituting each blood sugar profile.

According to an embodiment, when the blood sugar profile is stored in units of one week, the electronic apparatus 100 obtains blood sugar information measured at times corresponding to each other, of the plurality of blood sugar information constituting each blood sugar profile. Thereafter, the electronic apparatus 100 may determine the blood sugar information corresponding to a predetermined condition, of the blood sugar information measured at times corresponding to each other, and generate the blood sugar history profile 620 using that determined blood sugar information.

Here, the predetermined condition may be, for example, a condition for determining the blood sugar information measured for the most number of times, of the blood sugar information measured at times corresponding to each other, as the blood sugar information for constituting the blood sugar history profile 620.

When the blood sugar history profile 620 is generated through such various embodiments, the electronic apparatus 100 analyzes the blood sugar history profile 620 based on the predetermined normal blood sugar section and determines whether or not there is a section that is outside the normal blood sugar section.

For example, of the plurality of blood sugar information constituting the blood sugar history profile 620, the blood sugar numerical value corresponding to the blood sugar information of 17:00 to 19:00 may be lower than the predetermined low blood sugar numerical value (70 mg/dL) 610. In this case, the electronic apparatus 100 may predict that the user's blood sugar falls to low blood sugar between 17:00 to 19:00. Therefore, based on the predicted analysis result, the electronic apparatus 100 may provide blood sugar care information that blood sugar care is needed between 17:00 to 19:00, which is before the time it was predicted that the blood sugar will fall to a low state.

Meanwhile, when the blood sugar history profile 620 is generated, the electronic apparatus 100 may generate a blood sugar history profile section 630 based on the plurality of blood sugar information constituting the generated blood sugar history profile 620. For example, the electronic apparatus 100 may set each blood sugar information constituting the blood sugar history profile 620 as an intermediate value, determine an error range based on the blood sugar information set as the intermediate value, and generate the blood sugar history profile section 630 based on the determined error range. For example, when the value of the blood sugar information corresponding to 12:00, of the blood sugar information constituting the blood sugar history profile 620, is 100 mg/dL, the error range may be, with reference to 100 mg/dL, maximum blood sugar information being 110 mg/dL and minimum blood sugar information being 90 mg/dL.

When the blood sugar history profile section 630 is generated through such an embodiment, the electronic apparatus 100 may analyze the blood sugar history section 630 with reference to a predetermined low blood sugar numerical value (70 mg/dL) 610 and determine whether or not there is a section outside the determined low blood sugar numerical value (70 mg/dL) 610.

When there is a section outside the predetermined low blood sugar numerical value (70 mg/dL) 610, the electronic apparatus 100 may determine that blood sugar was not taken care of well at the time corresponding to the corresponding section. Therefore, based on such blood sugar history profile section 630, the electronic apparatus 100 may provide blood sugar care information that blood sugar care is needed prior to the time corresponding to the section that is outside the predetermined low blood sugar numerical value (70 mg/dL) 610.

For example, of the plurality of blood sugar information constituting the blood sugar history profile section 630, the blood sugar numerical value corresponding to the blood sugar information between 16:00 to 20:00 may be lower than the predetermined low blood sugar numerical value (70 mg/dL) 610. In this case, the electronic apparatus 100 may predict that the user's blood sugar will fall between 16:00 and 20:00. Therefore, based on the predicted analysis result, the electronic apparatus may provide blood sugar care information that blood sugar care is needed between 16:00 to 20:00 which is prior to the time predicted that the blood sugar will fall to a low state.

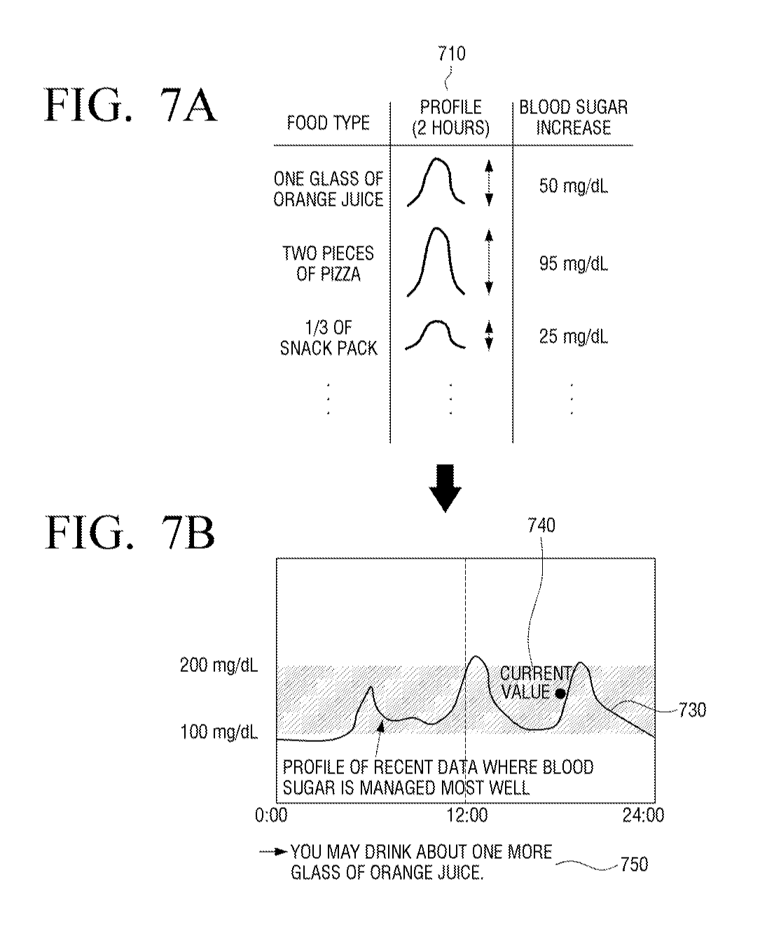

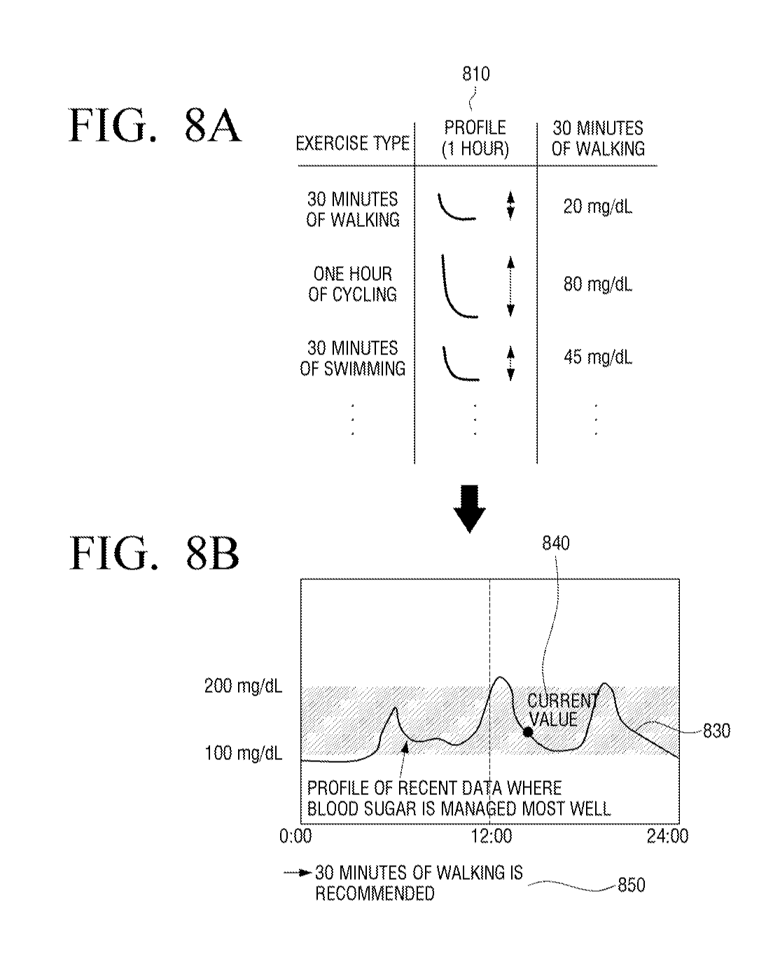

FIGS. 7A and 7B are first views for providing blood sugar care service based on activity information of a user in an electronic apparatus according to various embodiments of the present disclosure, and FIGS. 8A and 8B are second views for providing blood sugar care service based on activity information of a user in an electronic apparatus according to various embodiments of the present disclosure.

The electronic apparatus 100 may not only receive input of blood sugar information regularly measured by the blood sugar measurement apparatus (not illustrated), but also activity information according to life of the user, from the user.

Referring to FIG. 7A, the electronic apparatus 100 may receive food information included in the activity information of the user. In this case, together with the type and time of food that the user took based on the input food information, the electronic apparatus 100 may match diet care information 710 where the blood sugar profile and blood sugar change information according to the blood sugar information measured based on the corresponding food intake time are matched, and store the same.

For example, when food information indicating that the user took one glass of orange juice is input, the electronic apparatus 100 may determine the amount of change of blood sugar (50 mg/dL) based on the blood sugar information measured at the time the user drank the orange juice (2 hours) and the blood sugar information measured before drinking the orange juice.

When the amount of change of blood sugar is determined based on such food information of the user, the electronic apparatus 100 may match the diet care information 710 where the blood sugar profile state and the blood sugar change information during the 2 hours when the user drank the orange juice are matched, and store the same.

When the blood sugar information 740 measured of the current user is received from the blood sugar measurement apparatus (not illustrated) in a state where the diet care information 710 where the activity information including food information of the user and blood sugar state information according thereto are matched is stored, the electronic apparatus analyzes the blood sugar care state of the time where the blood sugar information 740 of the user was measured based on the predetermined target profile 730.

When it is determined that the blood sugar state of the current user is good based on the target profile 730, the electronic apparatus 100 may provide activity information 750 of the user based on the pre-stored diet care information 710.

For example, as illustrated in FIG. 7B, if the blood sugar information measured of the current user based on the target profile 730 is 150 mh/dL, the electronic apparatus 100 may display on the screen activity information 750 stating ‘you may drink about one more drink of orange juice’ based on the predetermined diet care information 710.

Accordingly, the user may take care of one's blood sugar by adjusting the diet based on the activity information 750 displayed on the screen of the electronic apparatus 100.

Referring to FIGS. 8A and 8B, the electronic apparatus 100 may receive input of motion information included in the activity information of the user. In this case, based on the exercise information input and together with the type and time of exercise that the user took, the electronic apparatus 100 may match the exercise care information 810 where the blood sugar profile and blood sugar change information according to the blood sugar information measured based on the corresponding exercise time are matched, and store the same.

For example, when exercise information on walking exercise is input, the electronic apparatus 100 may determine the amount of change of blood sugar (20 mg/dL) based on the blood sugar information measured during the time of the walking exercise (2 hours) and before taking the walking exercise based on the input exercise information.

When the amount of change of blood sugar is determined based on such user's exercise information, the electronic apparatus may match the exercise information of the walking exercise and the duration of the walking exercise, with the exercise care information 810 where the blood sugar profile state and the blood sugar change information during the 2 hours of walking exercise were matched, and store the same.

When the blood sugar information 840 measured of the current user is received from the blood sugar measurement apparatus (not illustrated) in a state where the exercise care information 810 where the activity information including the exercise information of the user and the blood sugar state information according thereto are matched is stored, the electronic apparatus 100 analyzes the blood sugar care state of the time the current user's blood sugar information 840 was measured with reference to the predetermined target profile 830.

When it is determined that the state of blood sugar of the current user is good with reference to the target profile 830, the electronic apparatus 100 may provide the activity information 850 of the user based on the pre-stored diet care information 810.

For example, as illustrated in FIG. 8B, if the blood sugar information measured of the current user with reference to the target profile 830 is 130 mh/dL, the electronic apparatus 100 may display the activity information 810 stating ‘30 minutes of walking is recommended’ on the screen based on the pre-stored exercise care information 810.

Accordingly, the user may take care of one's blood sugar by exercising based on the activity information 850 displayed on the screen of the electronic apparatus 100.

So far, operations for providing blood sugar care service of a user in the electronic apparatus 100 according to the present disclosure were explained. Hereinafter, a method for providing blood sugar care service of a user in the electronic apparatus 100 according to an embodiment of the present disclosure will be explained.

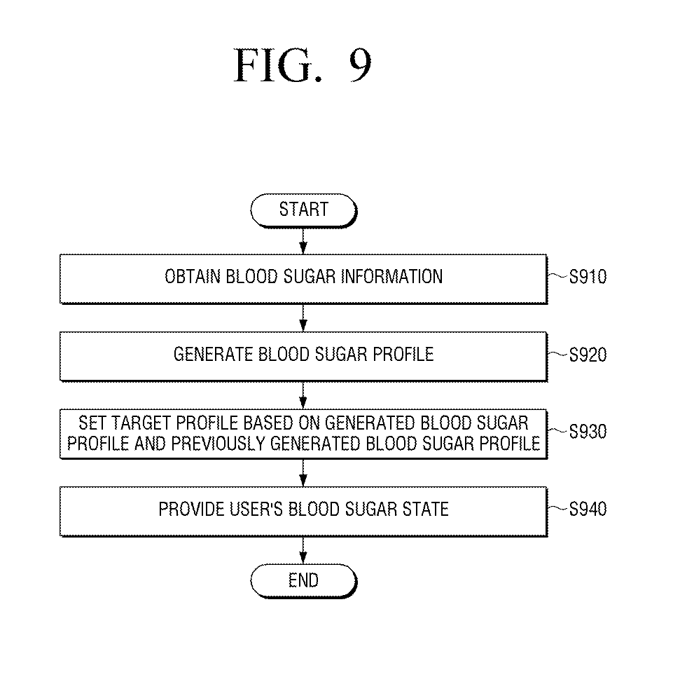

FIG. 9 is a flowchart of a method for providing blood sugar care service in an electronic apparatus according to an embodiment of the present disclosure.

Referring to FIG. 9, the electronic apparatus 100 obtains blood sugar information regularly measured of the user at operation S910.

According to an embodiment, the electronic apparatus 100 may be attached to the user's body and regularly measure the blood sugar of the user and obtain blood sugar information of the user.

According to an embodiment, the electronic apparatus 100 may perform communication with the blood sugar measurement apparatus (not illustrated) attached to the user's body and configured to regularly measure the blood sugar of the user, and receive the measured blood sugar information from the blood sugar measurement apparatus (not illustrated).

When the user's blood sugar information is obtained through such various embodiments, the electronic apparatus 100 collects the obtained blood sugar information during a predetermined time unit, and generates a blood sugar profile based on the blood sugar information collected for the predetermined time unit at operation S920.

Thereafter, the electronic apparatus 100 compares a currently generated blood sugar profile and a pre-generated blood sugar profile, and sets the blood sugar profile proximate to a predetermined normal blood sugar range as a target profile at operation S830. Here, the normal blood sugar range may be a blood sugar range section that is a medical standard. Further, of the two blood sugar profiles, the blood sugar profile proximate to the normal blood sugar range may be a blood sugar profile that belongs to the normal blood sugar range or that is close to the normal blood sugar range.

When such a target profile is set, the electronic apparatus 100 provides the blood sugar state of the user based on a predetermined target profile at operation S940.

Hereinafter, the method for setting the target profile will be explained in more detail.

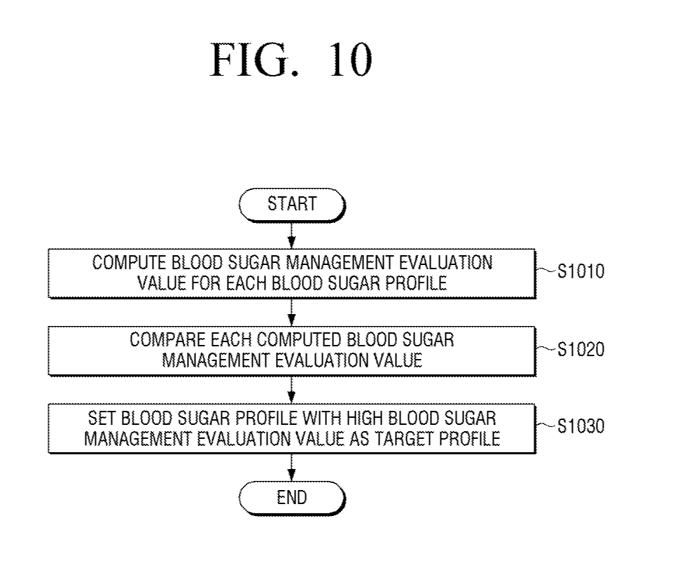

FIG. 10 is a flowchart for setting a target profile in an electronic apparatus according to an embodiment of the present disclosure.

Referring to FIG. 10, according to a predetermined condition, the electronic apparatus 100 computes a blood sugar care evaluation value corresponding to each of the previously generated blood sugar profile (hereinafter referred to as a first blood sugar profile) and the currently generated blood sugar profile (hereinafter referred to as a second blood sugar profile) at operation S1010.

For example, the electronic apparatus 100 may compute the blood sugar evaluation value corresponding to each of the first and the second blood sugar profile based on at least one of the times the blood sugar information is maintained outside the normal blood sugar range based on the information on where the blood sugar profile is included within the normal blood sugar range, the difference between the maximum blood sugar value and the minimum blood sugar value based on the plurality of blood sugar information included in the blood sugar profile and the information on the plurality of blood sugar information included in the blood sugar profile.

For example, to the times where the blood sugar information is maintained outside the normal blood sugar range, different weighted values may be applied based on the information on where the blood sugar profile corresponding to a first condition is included in the normal blood sugar range, difference between the maximum blood sugar value and the minimum blood sugar value based on the plurality of blood sugar information included in the blood sugar profile corresponding to a second condition, and the plurality of blood sugar information included in the blood sugar profile corresponding to a third condition.

As the method for computing the blood sugar evaluation value corresponding to each of the first and the second blood sugar profile according to the predetermined condition was explained, a detailed explanation will be omitted hereinafter.

When the blood sugar care evaluation value corresponding to each of the first and the second blood sugar profile is computed, the electronic apparatus 100 determines that the blood sugar profile having a higher value, of the computed blood sugar care evaluation values corresponding to each of the first and the second blood sugar profile, is a blood sugar profile proximate to the predetermined normal blood sugar range at operation S1020, and sets the corresponding blood sugar profile as a target profile at operation S1030.

That is, the electronic apparatus 100 may determine that the blood sugar profile having a higher value, of the blood sugar care evaluation values corresponding to each of the first and the second profile, is the blood sugar profile that belongs to the normal blood sugar range or the blood sugar profile proximate to the normal blood sugar range, and set the corresponding blood sugar profile as the target profile.

After the target profile is set through such an embodiment, when the blood sugar profile is generated based on the plurality of blood sugar information obtained for the predetermined period of time, the electronic apparatus 100 computes the blood sugar care evaluation value of the currently generated blood sugar profile as mentioned above. Thereafter, the electronic apparatus 100 may compare the blood sugar care evaluation value computed from the currently generated blood sugar profile and the previously computed blood sugar care evaluation value regarding the blood sugar profile set as the target profile, and re-set the blood sugar profile having a higher value, of the two evaluation values, as the target profile.

Meanwhile, after the target profile is set through the aforementioned embodiment, when the blood sugar information is obtained, the electronic apparatus 100 may provide the state of blood sugar of the user through the following embodiments.

According to an embodiment, after the target profile is set, when the blood sugar information measured of the user is obtained, the electronic apparatus 100 may compare the obtained blood sugar information and the blood sugar information of the time point the current blood sugar was obtained, of the plurality of blood sugar information constituting the target profile, and provide the current blood sugar state of the user.

According to another embodiment, after the target profile is set, when the blood sugar profile is generated based on the measured blood sugar information, the electronic apparatus 100 may analyze the currently generated blood sugar profile based on at least one of the target profile and the normal blood sugar range, and provide the state of blood sugar of the user by period.

For example, the electronic apparatus 100 may provide the state of blood sugar of the user by period through the following embodiments.

According to an embodiment, of a plurality of predetermined evaluation grade sections, the electronic apparatus 100 may provide the blood sugar care state of the user by period based on the evaluation grade section where the extent (distribution)(%) where the currently generated blood sugar profile belongs to the normal blood sugar range or the error extent (%) between the currently generated blood sugar profile and the target profile belongs to.

For example, the currently generated blood sugar profile may belong to the normal blood sugar range, and the error range between the currently generated blood sugar profile and the predetermined target profile may be less than 20%. In this case, the electronic apparatus 100 may determine that the user's blood sugar care belongs to a first evaluation grade indicating a good state, and provide the state of blood sugar of the user by period according thereto.

Meanwhile, the currently generated blood sugar profile may not belong to the normal blood sugar range and the error range between the currently generated profile and the predetermined target profile may be less than 50%. In this case, the electronic apparatus 100 may determine that the user's blood sugar care belongs to a second evaluation grade indicating a state where blood sugar care is needed, and provide the state of blood care of the user by period according thereto.

Meanwhile, the currently generated blood sugar profile may be lower or higher than the abnormal blood sugar value indicating a low blood sugar or a high blood sugar, and the error range between the currently generated blood sugar profile and the predetermined target profile may be 50% or above. In this case, the electronic apparatus 100 may determine that the user's blood sugar care belongs to a third evaluation grade indicating that the user's blood sugar is not taken care of, and provide the state of blood sugar care of the user by period, according thereto.

According to an additional aspect of the present disclosure, the electronic apparatus 100 may generate a blood sugar history profile based on the blood sugar information included in each of the plurality of blood sugar profiles generated in predetermined times units.

When such a blood sugar history profile is generated, the electronic apparatus 100 determines the section that is outside the normal blood sugar range based on the generated blood sugar history profile. Thereafter, the electronic apparatus 100 may predict the period where the user's blood sugar will go outside the normal blood sugar range based on the section determined as existing outside the normal blood sugar range.

According to an additional aspect of the present disclosure, when activity information is input by the user, the electronic apparatus 100 stores the input activity information and the blood sugar information measured at the time point the corresponding activity information was generated. Here, the activity information may include at least one of the information on food taken by the user and information on exercise taken by the user. Further, the time point where the activity information was generated may be the time the food was taken by the user and the time the exercise was taken by the user.

Therefore, after the target profile is set, when the blood sugar information measured of the user is obtained, the electronic apparatus 100 may provide the pre-stored activity information based on the blood sugar information of the point where the pre-obtained blood sugar information is located on the target profile.

According to an additional aspect of the present disclosure, the electronic apparatus 100 transmits the blood sugar profile generated in predetermined time units to the external server (not illustrated). Here, the external server (not illustrated) generates ranking information of the blood sugar care group to which the corresponding user belongs based on the blood sugar profile of the user received from the electronic apparatus 100.

For example, when the blood sugar profile of the user is received from the electronic apparatus 100, the external server (not illustrated) may compare and analyze the blood sugar profile of the corresponding user and the blood sugar profiles of the users in the blood sugar care group that the corresponding user belongs to and determine a ranking in the order of blood sugar profiles most proximate to the predetermined normal blood sugar section, and transmit the ranking information of the corresponding user to the electronic apparatus 100. Accordingly, the electronic apparatus 100 may provide the ranking information of the blood sugar care group received from the external server (not illustrated).

Meanwhile, the method for providing blood sugar care service according to the aforementioned various embodiments may be coded in software and stored in a non-transitory readable medium. Such a non-transitory readable medium may be mounted onto various apparatuses and be used.

The term “non-transitory computer readable medium” refers not to a medium that stores data for a short period of time such as a register, cache, memory but a medium readable by devices and that stores data semi-permanently. For example, it may be a compact disc (CD), digital versatile disc (DVD), hard disk, blue ray disk, USB, memory card, and ROM, etc.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A method for providing blood sugar care service in an electronic apparatus, the method comprising:
obtaining blood sugar information of a user;
when the blood sugar information is obtained for a predetermined time unit, generating a blood sugar profile based on the blood sugar information obtained during the predetermined time unit;
comparing the generated blood sugar profile and a previously generated blood sugar profile;
setting a blood sugar profile proximate to a normal blood sugar range as a target profile; and
providing a state of blood sugar of the user based on the set target profile.

2. The method according to claim 1, wherein the setting of the target profile comprises:
computing a blood sugar care evaluation value corresponding to each of the generated blood sugar profile and the previously generated blood sugar profile, according to a predetermined condition;
determining the blood sugar profile having a higher value, of the computed blood sugar care evaluation values, as the blood sugar profile proximate to the predetermined normal blood sugar range; and
setting the determined blood sugar profile as the target profile.

3. The method according to claim 2,
wherein the computing computes the blood sugar care evaluation value of the blood sugar profile based on at least one of an extent to which the blood sugar profile is included in the normal blood sugar range, a difference between a maximum blood sugar value and a minimum blood sugar value based on a plurality of blood sugar information included in the blood sugar profile, or a period of time the blood sugar information is maintained outside the normal blood sugar range based on the plurality of blood sugar information included in the blood sugar profile.

4. The method according to claim 1, wherein, when the blood sugar information is obtained after the target profile is set, the providing comprises:
comparing the blood sugar information at the time point the blood sugar information was received, of the plurality of blood sugar information constituting the target profile, and the received blood sugar information; and
providing a current blood sugar state of the user.

5. The method according to claim 1, wherein, when the blood sugar profile is generated based on the obtained blood sugar information after the target profile is set, the providing comprises:
analyzing the generated blood sugar profile based on at least one of the target profile and the normal blood sugar range; and
providing a state of blood sugar care of the user by period.

6. The method according to claim 5,
wherein the providing provides the state of blood sugar care of the user by period based on an extent where the generated blood sugar profile belongs to the normal blood sugar range, of a plurality of predetermined evaluation grade sections, or the evaluation grade section to which an error extent between the generated blood sugar profile and the target profile belongs.

7. The method according to claim 1,
further comprising generating a blood sugar history profile based on a plurality of blood sugar information included in each of a plurality of blood sugar profiles generated in the predetermined time unit,
wherein the providing comprises:
determining a section that exists outside the normal blood sugar range based on the blood sugar history profile;
predicting a period where the user's blood sugar will be outside the normal blood sugar range based on the determined section; and
providing the predicted period.

8. The method according to claim 1,
further comprising, when activity information including at least one of food information and exercise information of the user is input, storing the activity information and the blood sugar information measured at the time point the activity information was generated,
wherein, when the blood sugar information measured of the user is obtained after the target profile is generated, the providing provides the pre-stored activity information based on the blood sugar information of the point where the obtained blood sugar information is located on the target profile.

9. The method according to claim 1, further comprising:
transmitting the blood sugar profile to an external server; and
when the external server generated and transmitted ranking information of a blood sugar care group to which the user belongs based on the blood sugar profile, receiving and providing the ranking information.

10. The method according to claim 1, wherein the blood sugar information of the user is obtained by measuring on a periodic basis.

11. An electronic apparatus comprising:
a blood sugar measurer configured to measure blood sugar of a user;
an outputter configured to output a state of blood sugar of the user; and
a processor configured, when blood sugar information measured by the blood sugar measurer is obtained during a predetermined time unit, to:
generate a blood sugar profile based on the blood sugar information obtained during the predetermined time unit,
compare the generated blood sugar profile and a previously generated blood sugar profile,
set a blood sugar profile proximate to a normal blood sugar range as a target profile, and
control the outputter to output a state of blood sugar of the user based on the set target profile.

12. The electronic apparatus according to claim 11, wherein the processor is further configured to:
compute a blood sugar care evaluation value corresponding to each of the generated blood sugar profile and the previously generated blood sugar profile according to a predetermined condition,
determine the blood sugar profile having a higher value, of the computed blood sugar care evaluation values, as the blood sugar profile proximate to the predetermined normal blood sugar range, and
set the determined blood sugar profile as the target profile.

13. The electronic apparatus according to claim 12,
wherein the processor is further configured to compute the blood sugar care evaluation value of the blood sugar profile based on at least one of an extent to which the blood sugar profile is included in the normal blood sugar range, a difference between a maximum blood sugar value and a minimum blood sugar value based on a plurality of blood sugar information included in the blood sugar profile, or a period of time the blood sugar information is maintained outside the normal blood sugar range based on the plurality of blood sugar information included in the blood sugar profile.

14. The electronic apparatus according to claim 11, wherein, when the blood sugar information is obtained after the target profile is set, the processor is further configured to:
compare the blood sugar information at the time point the blood sugar information was received, of the plurality of blood sugar information constituting the target profile, and the received blood sugar information, and
control the outputter to provide a current blood sugar state of the user.

15. The electronic apparatus according to claim 11, wherein, when the blood sugar profile is generated based on the obtained blood sugar information after the target profile is set, the processor is further configured to control the outputter to:
analyze the generated blood sugar profile based on at least one of the target profile and the normal blood sugar range, and
output a state of blood sugar care of the user by period.

16. The electronic apparatus according to claim 15,
wherein the processor is further configured to provide the state of blood sugar of the user by period based on an extent the generated blood sugar profile belongs to the normal blood sugar range of a plurality of predetermined evaluation grade sections or the evaluation grade section to which an error extent between the generated blood sugar profile and the target profile belongs.

17. The electronic apparatus according to claim 15, wherein the processor is further configured to:
generate a blood sugar history profile based on a plurality of blood sugar information included in each of a plurality of blood sugar profiles generated in the predetermined time unit,
determine a section that exists outside the normal blood sugar range based on the blood sugar history profile,
predict a period where the user's blood sugar will be outside the normal blood sugar range based on the determined section, and
control the outputter to output the predicted result information.

18. The electronic apparatus according to claim 11, further comprising a storage, wherein,
when activity information including at least one of food information and exercise information of the user is input, the processor is further configured to control the storage to store the activity information and the blood sugar information measured at a time point the activity information was generated, and
when the blood sugar information measured of the user is obtained after the target profile is generated, the processor is further configured to control the outputter to output the activity information stored in the storage based on the blood sugar information at a point where the obtained blood sugar information is located on the target profile.

19. The electronic apparatus according to claim 11, further comprising a communicator configured to transmit the blood sugar profile to an external server, wherein,
when the external server generated and transmitted ranking information of a blood sugar care group to which the user belongs based on the blood sugar profile, the processor is further configured to control the outputter to output the received ranking information when the ranking information is received.

20. A computer readable recording medium storing a program for executing the following operations in combination with an electronic apparatus, the operations comprising:
obtaining blood sugar information of a user;
when the blood sugar information is obtained during a predetermined time unit, generating a blood sugar profile based on the blood sugar information obtained during the predetermined time unit;
comparing the generated blood sugar profile and a previously generated blood sugar profile;
setting a blood sugar profile proximate to a normal blood sugar range as a target profile; and
providing a state of blood sugar of the user based on the set target profile.
